(19) United States
(12) Patent Application Publication
(10) Pub. No.: US 2024/0252431 A1
(43) Pub. Date: Aug. 1, 2024

(54) PULMONARY BIGUANIDE FORMULATIONS AND DELIVERY DEVICES

(71) Applicant: Onconox Inc., Monmouth Junction, NJ (US)

(72) Inventor: Sitaram Velaga, Monmouth Junction, NJ (US)

(73) Assignee: Onconox Inc., Monmouth Junction, NJ (US)

(21) Appl. No.: 18/561,168

(22) PCT Filed: Dec. 13, 2021
(86) PCT No.: PCT/US2021/072881

§ 371 Date: Nov. 15, 2023

Related U.S. Application Data
(60) Provisional application No. 63/223,622, filed on Jul. 20, 2021.

(51) Int. Cl. A61K 9/00; A61K 31/155; A61M 11/00

(52) U.S. Cl. CPC A61K 9/0078; A61K 9/0043; A61K 31/155; A61M 11/006

(57) ABSTRACT

Compositions for pulmonary delivery comprising a biguanide or a pharmaceutically acceptable salt thereof in a form suitable to be aerosolized for pulmonary delivery to a human subject, and methods of treating lung disease.

### FIELD OF SUBJECT DISCLOSURE

Pulmonary formulations and delivery of Biguanide, such as an adjunct for sensitization of standalone radiation, or in a concurrent chemotherapy or monoclonal antibodies and biological inhibitors in lung cancers, including small lung cancer lung cancer (SCLC) Patients and non-small lung cancer lung cancer (NSCLC) Patients.

### BACKGROUND OF THE DISCLOSURE

In the United States, lung cancer continues to be one of the leading causes of death in cancers (Duma et al., 2019). Lung cancer comprises of two categories: a) non-small lung cancer (NSCLC (approximately 85% of cases) and small cell lung cancer (SCLC) (approximately 15%). The WHO has classified NSCLC into 3 main types: adenocarcinoma, squamous cell carcinoma, and large cell. There are also several variants and combinations of clinical subtypes. The 60-month overall survival rate for NSCLC remains poor, from 68% in patients with stage IB disease to 0% to 10% in patients with stage IVA-IVB disease (Goldstraw et al., 2016). The National Lung Screening Trial found a lung cancer mortality benefit of 20%, and a 6.7% decrease in all-cause mortality, with the use of low-dose chest computed tomography in the suspected high-risk individuals (Duma et al., 2019).

Surgery is the recommended treatment for patients with stage I-II non-small-cell lung cancer (NSCLC) (Hirsch et al., 2019). Depending on the clinical stage of the patients, a 5-year survival rate for clinical stage IA, stage IB, stage IIA, and stage IIB are 77-92%, 68%, 60% and 53%, respectively (Hirsch et al., 2019). For patients with good performance status that present locally advanced NSCLC (stages IIIA-B) which is not amenable to surgical resection, the current standard of care involves a 6-week course of thoracic radiotherapy with the concurrent delivery of doublet chemotherapy using either cisplatin or carboplatin and a second drug per week or every 3 weeks (Hirsch et al., 2017). The treatment landscape for treating advanced and metastatic lung cancer has greatly evolved in the last decade with the introductions of molecular based therapies which are aimed to target specific mutations that occur in the patients (Hirsch et al., 2017).

Radiation Therapy (RT) delivers high-energy X-rays that can destroy rapidly dividing cancer cells or to palliate symptoms. The role of curative-intent RT is well established in locally advanced and early-stage NSCLC (Baker et al., 2016). In addition to being used as a primary treatment, RT can shrink the tumor prior to surgery and post-surgery, and can eliminate residual cancer cells that remain in the treated area. However, the delivery of RT to thorax remains a significant owing to the low electron density of lung, respiratory- and cardiac-induced tumor motion, as well as proximity to critical structures such as the esophagus and spinal cord. While advanced RT technologies like stereotactic ablative radiotherapy (SABR) can address many of these challenges (Baker et al., 2016). SABR is now considered the standard of care for medically inoperable patients with peripheral early-stage NSCLC. Also, toxicity associated with peripheral lung SABR delivery appears to be modest (Baker et al., 2016). Recently, the combined roles of RT with immunotherapy based on checkpoint inhibitors has received attention in NSCLC. There has been a suggestion that two treatment modalities may combine to offer synergistic responses for NSCLC.

There nevertheless remains a need to improve the effectiveness of current therapies for lung cancers, such as NSCLC and SCLC. There also remains a need to prevent side effects attendant to radiation therapy, such radiation induced lung injury, including radiation pneumonitis and radiation fibrosis.

### SUMMARY

One exemplary embodiment of the subject disclosure provides a composition for pulmonary delivery comprising a biguanide (e.g., metformin) or a pharmaceutically acceptable salt thereof (e.g., metformin hydrochloride) in a form suitable to be aerosolized for pulmonary delivery to a human subject.

Another exemplary embodiment of the subject disclosure provides a pulmonary delivery system for delivery of a biguanide comprising a biguanide or a pharmaceutically acceptable salt thereof in a form suitable to be aerosolized for pulmonary delivery to a human subject and a nebulizer.

Another exemplary embodiment of the subject disclosure provides a method of treating a lung condition or disease in a subject comprising administering to the subject via the pulmonary route a composition comprising a biguanide or a pharmaceutically acceptable salt thereof.

Yet another exemplary embodiment of the subject disclosure provides a method of treating lung cancer in a subject comprising administering to the subject (a) via the pulmonary route a composition comprising a biguanide or a pharmaceutically acceptable salt thereof and (b) administering a round of radiation therapy to the subject.

Yet another exemplary embodiment of the subject disclosure provides a method of treating lung cancer in a subject comprising administering to the subject (a) via the pulmonary route a composition comprising a biguanide or a pharmaceutically acceptable salt thereof and (b) administering a round of chemotherapy to the subject.

Yet another exemplary embodiment of the subject disclosure provides a method of treating lung cancer in a subject comprising administering to the subject (a) via the pulmonary route a composition comprising a biguanide or a pharmaceutically acceptable salt thereof and (b) administering a round of a monoclonal antibody to the subject.

Yet another exemplary embodiment of the subject disclosure provides a method of treating lung cancer in a subject comprising administering to the subject (a) via the pulmonary route a composition comprising a biguanide or a pharmaceutically acceptable salt thereof and (b) administering a round of small molecule tyrosine kinase inhibitor to the subject.

A method of preventing or treating radiation induced lung injury in a subject comprising administering to the subject via the pulmonary route a composition comprising a biguanide or a pharmaceutically acceptable salt thereof. Radiation induced lung injuries include radiation pneumonitis and radiation fibrosis

### BRIEF DESCRIPTION OF THE FIGURES

The accompanying drawings, which are incorporated in and constitute a part of the specification, illustrate embodiments of the subject disclosure and, together with the description, explain principles of the subject disclosure.

FIG. 1 is a schematic of the components of an exemplary nebuliser system for use in accordance with exemplary embodiments of the subject disclosure—the eFlow® rapid Nebuliser System from PARI GmbH;

FIG. 2 is a schematic of the components of an exemplary portable mesh nebulizer for use in accordance with exemplary embodiments of the subject disclosure—the InnoSpire Go available from Philips N.V.;

FIG. 3 is a schematic of the components of an exemplary disposable soft mist nebulizer—Pulmospray® from Medspray;

FIGS. 4A and 4B depict the Aerodynamic Particle Size Distribution (APSD) of metformin formulations as described in the Examples, FIG. 4A (5%) and FIG. 4B (10%) stage by stage as % of total dose; (squares—InnoSpire; diamonds—eFlow®; triangles—Pulmospray); and

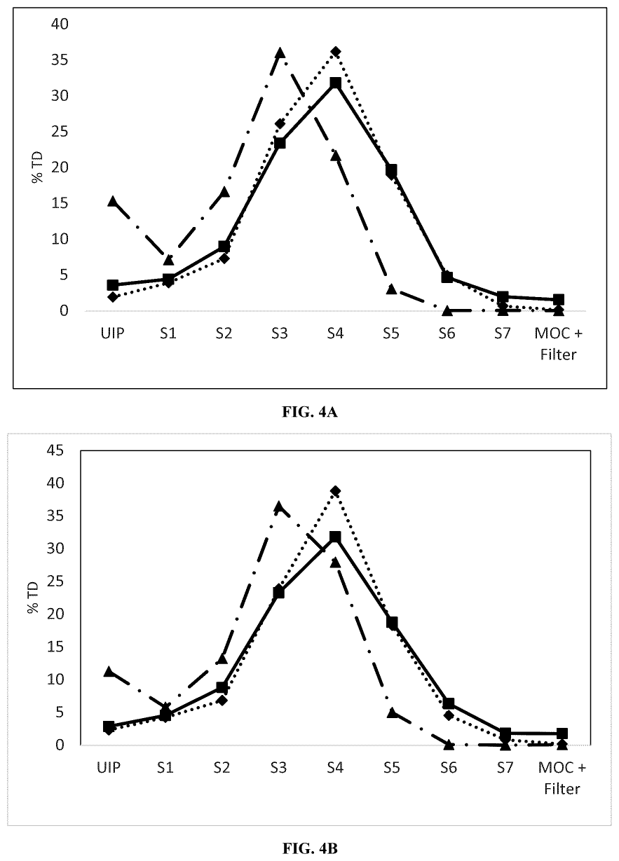

FIGS. 5A and 5B depict the mean plasma (FIG. 5A), and lung concentration (FIG. 5B) curves of metformin following exposure via nose only inhalation for 60 minutes of 5% (G1) or 10% (G2) formulations in rats, as described in the Examples.

### DETAILED DESCRIPTION

The invention can be more fully appreciated by reference to the following description, including the Examples. Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In addition, the materials, methods, and examples are illustrative only and not intended to be limiting.

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value. Alternatively, with respect to biological systems or processes, the term “about” can mean within an order of magnitude, within 5-fold, or within 2-fold on either side of a value. Numerical quantities given herein are approximate unless stated otherwise, meaning that the term “about” or “approximately” can be inferred when not expressly stated.

To provide a more concise description, some of the quantitative expressions given herein are not qualified with the term “about.” It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity for which that could be obtained under the particular stoichiometric conditions. Concentrations that are given as percentages refer to mass ratios, unless indicated differently.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof, unless limitation to the singular is explicitly stated.

The terms “comprising” and “including” are used herein in their open, non-limiting sense. Other terms and phrases used in this document, and variations thereof, unless otherwise expressly stated, should be construed as open ended, as opposed to limiting. Thus, the term “example” is used to provide exemplary instances of the item in discussion, not an exhaustive or limiting list thereof. Similarly, adjectives such as “conventional,” “traditional,” “normal,” “criterion,” “known,” and terms of similar meaning should not be construed as limiting the item described to a given time period or to an item available as of a given time, but they should be read to encompass conventional, traditional, normal, or criterion technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. As will become apparent to one of ordinary skill in the art after reading this document, the illustrated embodiments and their various alternatives may be implemented without confinement to the illustrated examples.

The term “carrier” refers to an adjuvant, vehicle, or excipients, with which the compound is administered. In certain embodiments of this invention, the carrier is a solid carrier. Suitable pharmaceutical carriers include those described in Remington: The Science and Practice of Pharmacy, 21st Ed., Lippincott Williams & Wilkins (2005).

The term “dosage form,” as used herein, is the form in which the dose is to be administered to the subject or patient.

The term “pharmaceutically acceptable,” as used in connection with compositions of the invention, refers to molecular entities and other ingredients of such compositions that are physiologically tolerable and do not typically produce untoward reactions when administered to an animal (e.g., human) according to their intended mode of administration (e.g., oral or parenteral).

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluents to facilitate administration of an agent and that is compatible therewith. Suitable pharmaceutical carriers include those described in Remington: The Science and Practice of Pharmacy, 21st Ed., Lippincott Williams & Wilkins (2005).

As used herein, the term “inert” refer to any inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

As used herein, the term “disorder” is used interchangeably with “disease” or “condition”. For example, a neurological disorder also means a neurological disease or a neurological condition.

The terms “treat,” “treating,” and “treatment” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. These terms also include ameliorating a symptom of a disease (e.g., reducing the pain, discomfort, or deficit), wherein such amelioration may be directly affecting the disease (e.g., affecting the disease's cause, transmission, or expression) or not directly affecting the disease.

As used herein the term “droplet size distribution” or DSD is used to indicate the statistical frequency of droplets of certain size in a sample as determined by laser diffraction and as defined in ISO 9276-2:2014: Representation of results of particle size analysis—Part 2: Calculation of average particle sizes/diameters and moments from particle size distributions.

As used in the present disclosure, the term “effective amount” is interchangeable with “therapeutically effective amount” and means an amount or dose of a compound or composition effective in treating the particular disease, condition, or disorder disclosed herein, and thus “treating” includes producing a desired preventative, inhibitory, relieving, or ameliorative effect. In methods of treatment according to the invention, “an effective amount” of at least one compound is administered to a subject (e.g., a mammal). The “effective amount” will vary, depending on the compound, the disease (and its severity), the treatment desired, age and weight of the subject, etc.

As used herein, the phrase “in combination” refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

The terms “individual,” “subject,” and “patient” are used interchangeably herein and can be a vertebrate, in particular, a mammal, more particularly, a primate (including non-human primates and humans) and include a laboratory animal in the context of a clinical trial or screening or activity experiment. Thus, as can be readily understood by one of ordinary skill in the art, the compositions and methods of the present invention are particularly suited to administration to any vertebrate, particularly a mammal, and more particularly, a human.

As used herein, the term “monoclonal antibody” refers to a monoclonal antibody class of cancer therapy drugs known in the art that inhibit, for example, EGFR, VEGF-A, PD-L1, PD-L2, or that otherwise target the PD-1 receptor of lymphocytes or CTLA-4. Monoclonal antibodies include, but are not limited to cetuximab, bevacizumab, nivolumab, pembrolizumab, atezolizumab, and ipilimumab.

As used herein, the term “small molecule tyrosine kinase inhibitors” refers to a small molecule class of cancer therapy drugs known in the art that inhibit tyrosine kinases, such as, but not limited to, EGFR, HER2, ALK, ROS1, and HGFR. Small molecule tyrosine inhibitors include, but are not limited to, gefitinib, erlotinib, dacomitinib, osimertinib, crizotinib, ceritinib, and lorlatinib.

The disclosed subject matter delivers a biguanide via the pulmonary route. In certain embodiments, the biguanide is administered via use of a nebulizer.

In certain embodiments, the drug is a biguanide, which, as used herein, refers to a class of drugs that function as oral antihyperglycemic drug, traditionally used for diabetes mellitus or prediabetes treatment. Examples of biguanides include metformin, phenformin, buformin and HL156A (also known as IM156). In a preferred embodiment, the biguanide is metformin or a pharmaceutically acceptable salt, or polymorphs thereof (e.g., metformin hydrochloride). For the purposes of brevity, reference to “metformin” or “metformin hydrochloride” is also understood to constitute reference to any one of the biguanides disclosed instantly, even though not specifically identified in the particular passage of the instant disclosure.

In certain embodiments, the formulations of the subject disclosure can be a solution, suspension or a dry powder.

Advantages of the instantly disclosed formulations and delivery systems can, in certain embodiments, include one or more of: a) maximizing the local drug deposition and absorption in the tissue of interest; b) minimal systemic effects c) rapid absorption into the tissue because of the high surface area of the lungs; d) owing to minimal intracellular and extracellular drug-metabolizing enzyme activities, high bioavailability of drugs can be achieved in the lungs, e) circumventing the first pass metabolism effect.

In certain embodiments, the pulmonary delivery system of the drug is so designed such that it minimizes drug dose relative to oral approved dose of the drug, but achieves higher exposure as measured by the area under the tissue concentration versus time curve for a defined period of time at the site of action. The ensuing uptake of the drug into lung tissue is also achieved faster and higher using the pulmonary delivery system as compared to the dosing of the oral drug formulation which tends to have a lag time due to oral absorption. Therefore, the present disclosure aids in providing an adjuvant in radiation therapy which results in a more targeted radiation therapy with lower side effects.

In certain embodiments, the formulations of the subject disclosure are administered via a nebulizer, including a breath enhanced nebulizer, a breath activated nebulizer, hand-held nebulizer based on jet, ultrasound, vibrating mesh technology or a soft mist inhalers such as, but not limited to, Respimat® or Pulmospray®. For example, the nebulizers can include, but are not limited to, a nebulizer selected from LC plus or eFlow (PARI), Aeroeclipse (Trudell), and Phillips Innospire Go ((Philips Respironics), Aeroneb® range (Aerogen Inc.). Other nebulizers known in the art can find use according to the disclosed subject matter.

For example, the PARI eFlow® is a battery-operated, compact, portable nebulizer using the ODEM TouchSpray atomising head that consists of a membrane with 4,000 laser-drilled apertures surrounded by a piezoelectric actuator to generate aerosol. The InnoSpire Go is a general-purpose portable mesh device. Any functionally equivalent device to those specific devices mentioned herein may be employed in the subject disclosure. In other certain embodiments, a formulation of the instant subject disclosure is pulmonary administered in a device (e.g., a dry powder inhaler) besides a nebulizer.

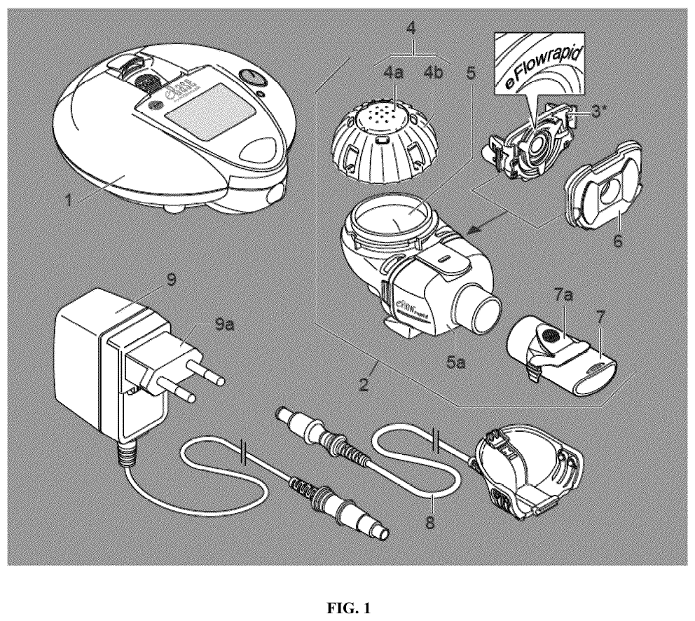

An exemplary an exemplary nebulizer system that can be used to administer formulations of the subject disclosure include the eFlow® rapid Nebuliser System from PARI GmbH, which is depicted in FIG. 1. According to this exemplary embodiment, the nebulizer system includes a controller (1) for controlling the device, a handset (2) which forms the base of the device, an aerosol head (3) which contains the vibrating mesh, a medication cap (4) which in turn is comprised of a caps seal (4a) and the cap (4b) itself, a medication reservoir (5) which holds the actual medication to be nebulized, a inspiratory valve (6) which acts as a check valve during inspiration, a mouth piece (7) comprising an expiratory valve (7a) which minimizes the loss of formulation during exhalation, a connection cord (8) and power adapter (9) which supplies power to the setup. In this device type, the aerosol is generated by a vibrating mesh operated at defined frequencies and amplitudes thereby by turning a liquid containing the medication into mist.

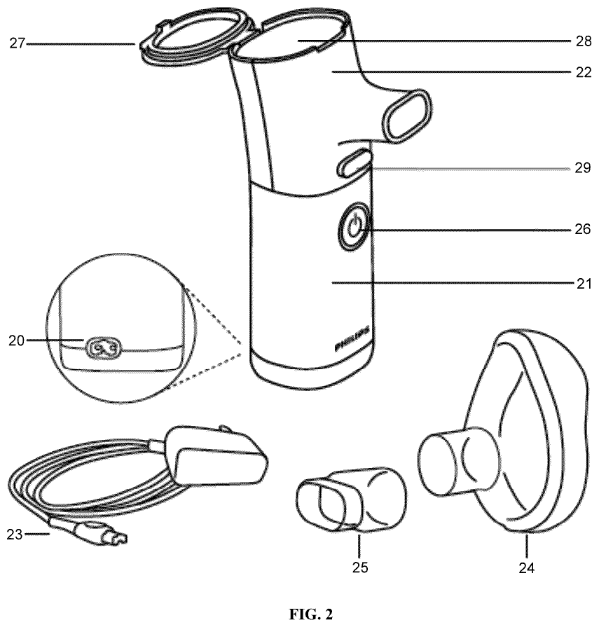

An exemplary mesh nebulizer that can be used to administer formulations of the subject disclosure include the InnoSpire Go portable mesh nebulizer available from Philips N. V. depicted in FIG. 2. According to this exemplary embodiment, the nebulizer includes a handset (21) which forms the base of the device, a mouth piece assembly (22) which contains the medication chamber (28) and medication lid (27) and serves as a reservoir for the medication to be nebulized, a power adapter (23) which is used for charging the device, a LiteTouch medium mask (age 1-5 years) (24), a mask adapter (25) which connects the mask to the mouthpiece, an On/Off button and LED indicator (26) to switch the device on or off or check the status of operation, a mouth piece assembly release button (29) which releases the mouth piece assembly during disassembling the device and a power socket (20) which connects the device to the power adapter. In this device type, the aerosol is generated by a vibrating mesh operated at defined frequencies and amplitudes thereby by turning a liquid containing the medication into mist.

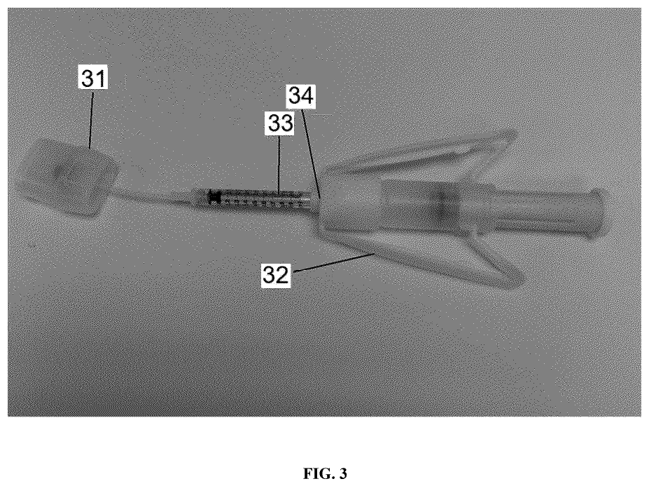

An exemplary soft mist nebulizer that can be used to administer formulations of the subject disclosure include the Pulmospray® from Medspray, which is depicted in FIG. 3. According to this exemplary embodiment, the nebulizer is disposable, and includes a mouth piece (31), actuation handles (32) which when used, force a predefined volume through the nozzle located in the mouth piece generating the aerosol, a medication reservoir (33) which holds the actual medication to be nebulized and a piston plunger (34) which seals the back end of the medication reservoir. This device types relies on the Rayleigh instability to create the aerosol. Here, the liquid is forced through small holes, forming jets which subsequently breakup in droplets which in turn form the aerosol.

In certain embodiments, the formulation includes an aqueous solution of a biguanide (e.g. metformin or pharmaceutically acceptable salt thereof). In certain embodiments the concentration (wt %) of biguanide (e.g., metformin or pharmaceutically acceptable salt thereof) can range, for example, from about 0.1% to about 50%, or from about 0.5% to about 25%, or from about 1% to about 15%, or from about 2.5% to about 10%.

In certain embodiments, the formulations contain excipients such as lactose, mannitol, buffer salts (citrate and phosphate), sodium chloride salt, and preservatives. In certain embodiments, the excipient is recognized as being safe for pulmonary administration to a human and/or have been approved by a regulatory authority for pulmonary delivery. The formulation can, in exemplary embodiments, be processed using either high shear or low shear mixtures and blenders. In certain embodiments the resulting formulations have an osmolality of 300-1500 mOsm/kg and pH 4.0-9.0, or pH 6.0-9.0

In certain embodiments, the formulations show metformin assay concentration>95% and total impurities<0.05% for a period of at least two years at long term stability condition (25° C./50% RH) or at least 6 months at accelerated condition (40° C./75% RH).

In certain embodiments, the formulation of biguanide (e.g., metformin or pharmaceutically acceptable salt thereof), when aerosolized using a device, emit a dose in which at least 25% of the emitted dose is contained in droplets with aerodynamic particle sizes of 1-5 microns.

In certain embodiments, the formulation of biguanide (e.g., metformin or pharmaceutically acceptable salt thereof), when aerosolized using a device, produce droplet size distributions (DSDs) of d10 1.8-4.5 μm, 3.8-8.4 μm, 7.2-15.8 μm. In certain embodiments, the corresponding MMADs<4.5 μm, GSD<2.0, FPD>25 mg, FPF>50%.

In certain embodiments, the formulation of metformin in a nebulizer delivers >30% of the nominal dose as per the USP<1601> test using an adult breathing pattern. In certain embodiments, the % emitted dose of nebulized formulation deposited in Next Generation Impactor (NGI) stages 4-6 i.e., size range 3.3 μm and 1.4 μm, is 30-70%.

In certain embodiments, the formulations are submitted to the pharmacokinetics and tissue distribution of metformin in male Wistar rats, through a nose-only dynamic inhalation chamber. Tₘₐₓ 30 min-1 h, Cₘₐₓ 300-600 μg/g lungs, and half-life 1.5 to 3 h for lungs, lung tissue to plasma concentration ratio 40 to 150 maintained up to at least 8 h following the inhaled drug delivery. In certain embodiments, the formulations show approximately dose proportional increase in peak plasma concentration and area under the curve in both matrices viz., plasma and lung.

In certain embodiments, the method of treatment include the nominal dose of the inhaled metformin formulations is 100 mg-3000 mg, and the local lung tissue levels of metformin in humans is between 2650 to 15000 ng/mL. The proposed doses can be, for example, administered 6-10 weeks in total to accommodate the entire single dosing cycle of the radiation, chemotherapy or other treatment regimen (e.g., monoclonal antibody or small molecule kinase inhibitors). In certain embodiments, for example, the metformin is inhaled 1-3 days prior to the (e.g.) radiation dose followed by 3-7 days between the radiation cycles and 1-3 days after last radiation cycle.

The inhaled (e.g., nebulized) product of metformin of the subject disclosure can be intended for local conditions/diseases in the lung, more specifically, in certain exemplary embodiments, as a sensitizer for standalone radiation therapy or in a concurrent chemotherapy or monoclonal antibodies and biological inhibitors in Non-Small Cell Lung Cancer (NSCLC) patients. Multiple mechanisms have been proposed to support the sensitization by metformin of radiation therapy and/or chemotherapy and/or other immuno-oncology therapy directed towards NSCLC treatment which includes inhibition of ATM-AMPK-p53/p21, inhibition of Akt-mTOR-4EBP1 pathways, reduction in angiogenesis with associated enhancement in expression of apoptosis markers (Brown et al., 2019; Yousef and Tsiani, 2017).

For example, for NSCLC, in particular locally advanced, unresectable NSCLC, radiation therapy in combination with concurrent chemotherapy (e.g., platinum-based chemotherapy agents such as cisplatin or carboplatin) followed by 12 months of maintenance therapy with Programmed death-ligand 1 (PD-L1) for ex. durvalumab. In exemplary, embodiments, the presently disclosed compositions comprising a biguanide or a pharmaceutically acceptable salt thereof can be administered in conjunction with each treatment regimen.

One embodiment of the present disclosure provides a method of preventing or treating radiation induced lung injury in a subject comprising administering to the subject via the pulmonary route a composition comprising a biguanide or a pharmaceutically acceptable salt thereof, such as metformin hydrochloride. Radiation pneumonitis and radiation fibrosis are two-dose limiting toxicities of radiation therapy (RT), particularly for lung cancer, estimated to occur in 5-20% of patients receiving RT. See Giuranno et al., Frontiers in Oncology, September 2019, Vol. 9, Art. 877. Metformin has been found to reverse established lung fibrosis in a mouse model for lung fibrosis elicited by the anti-cancer drug bleomycin, in which metformin treatment, starting three weeks after lung injury and continuing for five weeks, accelerated the resolution of well-established fibrosis. See Sunad Rangarajan et al., Metformin reverses established lung fibrosis in a bleomycin model. Nature Medicine, 2018; Ye Wang et al., Chinese Journal of Radiological Medicine and Protection; (12): 736-741, 2017. Similarly, metformin has been shown to protect against radiation induced pneumonitis. See Rasoul Azmoonfar et al., Adv. Pharm. Bull. 8(4):697-704 (2018); Marisol Arroyo-Hernandez et al., BMC Pulmonary Medicine, 21(9):2021. In certain embodiments, the biguanide can be administered before and/or after a round of RT, and can be administered prophylactically to prevent radiation induced injury from occurring, and/or administered after radiation induced lung injury has been suspected or diagnosed.

The subject disclosure provides following therapeutics benefits; a) Maximizing the local drug deposition and absorption in the tissue of interest; b) Minimal systemic effects c) Rapid absorption into the tissue because of the high surface area of the lungs; d) Owing to minimal intracellular and extracellular drug-metabolizing enzyme activities, high bioavailability of drugs in the lungs, e) Circumventing the first pass metabolism effect and avoiding gastrointestinal distress including abdominal pain, bloating, diarrhea caused by metformin.

### EXAMPLES

The following Examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1. Formulations

The composition of the metformin drug formulations (5% and 10% formulations based on metformin free base) is shown in Table 1.

TABLE 1
Composition of the drug product of
metformin, 5% and 10% formulations

| | | Quality/ | |
|---|---|---|---|
| Components | Function | Grade | Concentration (w/v) |
| Metformin | API | USP | 6.4% or 12.8% (HCl salt) |
| hydrochloride | | | 5% or 10% (free base) |
| NaOH/HCl | pH adjustment | USP | q.s. |
| Water for | Vehicle | USP | q.s. |
| injection | | | |

The final form of the drug product will be a sterile solution for oral inhalation in a pre-filled LDPE or glass ampoules having an accurate volume of formulation (e.g., 10% solution; 0.5, 1, 3, 5, 10 or 20 ml containing 50, 100, 300, 500, 1000, or 2000 mg per unit doses respectively). The physical properties tested the formulations include pH, osmolality which are in the range 6.5-8.5 and 693-1249 (mOsm/kg) respectively.

### Example 2. DSD of Different Concentrations and Devices

The DSD of the formulations of Example 1 was determined by a laser diffraction method using a Malvern Spraytec instrument equipped with an inhaler module. This module allows for the adaption of inhaler devices including nebulizers by using the universal induction port (UIP) also used for APSD testing. The DSD was assessed of nebulizers filled with 3 ml of formulation within the first 60 second of nebulization. The key descriptors were d10, d50, d90 and the span here defined as (d90−d10)/d50. Example results of two formulations (5% and 10%) and three different devices; Pari eFlow, Phillips Innospire Go, and Medspray's Pulmospray are shown in Table 2. The schematics of Pari eFlow, Phillips Innospire Go, and Medspray's Pulmospray are shown in FIGS. 1, 2 and 3 respectively, discussed above.

TABLE 2
Cumulative DSD of a 10% formulation in three different device

| | 5% Formulation | | | 10% Formulation | | |
|---|---|---|---|---|---|---|
| | | Innospire | Pulmo- | | Innospire | Pulmo- |
| Parameter | eFlow | Go | spray | eFlow | Go | spray |
| d10 | 2.12 | 1.87 | 4.50 | 2.17 | 1.80 | 4.35 |
| d50 | 4.19 | 4.04 | 8.64 | 4.03 | 3.67 | 8.38 |
| d90 | 7.70 | 8.12 | 15.82 | 7.23 | 7.27 | 15.43 |
| Span | 1.33 | 1.54 | 1.31 | 1.26 | 1.49 | 1.32 |

Replicate measurements (n = 3) performed and average data is presented, Determined by Malvern Spraytec

### Example 3. APSDs of Different Formulations and Devices as Determined Using NGI

The method utilizes the Next Generation Impactor (NGI) which is a cascade impactor. Testing conditions were set to comply to USP <1601> i.e. test flow rate was 15 liters per minute and the NGI was cooled to NMT 5° C. for at least 90 minutes to mitigate droplet evaporation during testing. In contrast to other inhalation devices, nebulizer does not have a metering mechanism. Rather, in the finished product the total dose is determined by the amount of the solution filled into the nebulizer which is subsequently used until exhaustion (sputtering). The fill level was set to 3 ml for eFlow and Innospire Go. The inhalation time was set to conventional 60 seconds to avoid streaking. The summary of APSD descriptors are shown in Table 3. As an example, APSDs as percentage of total dose recovered of 5% and 10% formulations using three different devices, Pari eFlow, Phillips Innospire Go, and Medspray's Pulmospray are shown in Tables 3 and 4 and FIG. 3.

TABLE 3
Summary descriptors of the APSD

| | | Size | | |
|---|---|---|---|---|
| Parameter | Definition | class | | Description |
| FPD | Fine particle dose, i.e. | <5 | μm | Inhalable fraction |
| | mass of drug <5 μm | | | |
| FPF | Fine particle fraction | <5 | μm | % of FPD |
| | | | | compared to total |
| | | | | dose ex device |
| MMAD | Mass Median | — | | Mode of the |
| | Aerodynamic Diameter | | | APSD |
| GSD | Geometric | — | | Span of APSD |
| | Standard Deviation | | | |
| Group 1 | Mouthpiece - Stage 1 | >14.1 | μm | Non inhalable |
| | | | | fraction |
| Group 2 | Stage 2-Stage 3 | 8.61-5.39 | μm | Upper airways |
| Group 3 | Stage 4-Stage 6 | 3.30-1.36 | μm | Lower airways |
| Group 4 | Stage 7 - Filter | <0.98 | μm | Ultrafines |

TABLE 4
Percentage of emitted dose deposited in different size classes of 5% and 10% formulations

| | | | | % of emitted dose | | | | | |
|---|---|---|---|---|---|---|---|---|---|
| | | | | | | Innospire | | | |
| | | Size | | eFlow | | Go | | Pulmospray | |
| Group | Definition | class | Description | 5% | 10% | 5% | 10% | 5% | 10% |
| Group | Mouthpiece- | >14.1 | Non | 6.6 | 5.8 | 7.4 | 8.0 | 17.1 | 22.5 |
| 1 | Stage 1 | μm | inhalable | | | | | | |
| | | | fraction | | | | | | |
| Group | Stage 2- | 8.61- | Upper | 30.8 | 33.4 | 32.1 | 32.4 | 49.8 | 52.7 |
| 2 | Stage 3 | 5.39 | airways | | | | | | |
| | | μm | | | | | | | |
| Group | Stage 4- | 3.30- | Lower | 61.7 | 60.0 | 56.9 | 56.2 | 33.0 | 24.8 |
| 3 | Stage 6 | 1.36 | airways | | | | | | |
| | | μm | | | | | | | |
| Group | Stage 7- | <0.98 | Ultrafines | 1.0 | 0.8 | 3.6 | 3.5 | 0.12 | 0.1 |
| 4 | Filter | μm | | | | | | | |

### Example 4. Delivered Dose Results

The delivered dose of the example formulations was determined as per USP<1601> using an adult breathing pattern for eFlow and Innospire Go devices using the 5% and 10% formulation. The adult breathing pattern was simulated by a breathing simulator (BRS 1100, Copley). The devices were filled with 3 ml. The delivered dose results for example 5% and 10% metformin formulations are presented in Table 5 and Table 6, respectively.

TABLE 5
Mass balance of delivered dose testing 5% metformin formulation

| | eFlow | | | Innospire Go | | |
|---|---|---|---|---|---|---|
| | | Mass of | | | Mass of | |
| | Volume | drug | % of | Volume | drug | % of |
| Parameter | (ml) | (mg) | nominal | (ml) | (mg) | nominal |
| Nominal dose | 3.0 | 155.0 | 100 | 3.0 | 155.0 | 100 |
| Dead volume | 1.1 | 54.3 | 35.0 | 0.7 | 33.6 | 21.7 |
| Nebulized | 2.0 | 103.4 | 66.7 | 2.4 | 124.1 | 80.1 |
| Total delivered dose | 1.2 | 60.8 | 39.2 | 1.2 | 61.0 | 39.4 |
| Formulation lost | 0.8 | 41.4 | 26.7 | 1.2 | 62.0 | 40.0 |
| during exhalation | | | | | | |
| Dose Inhalation/ | 1.9 | | | 1.0 | | |
| exhalation ratio | | | | | | |

Replicate measurements (n = 2) performed and average data is presented, as per USP<1601>

TABLE 6
Mass balance of delivered dose testing 10% metformin formulation

| | eFlow | | | Innospire Go | | |
|---|---|---|---|---|---|---|
| | | Mass of | | | Mass of | |
| | Volume | drug | % of | Volume | drug | % of |
| Parameter | (ml) | (mg) | nominal | (ml) | (mg) | nominal |
| Nominal dose | 3.0 | 288.3 | 100.0 | 3.0 | 288.3 | 100.0 |
| Dead volume | 0.9 | 81.7 | 28.3 | 0.6 | 57.7 | 20.0 |
| Nebulized | 2.2 | 206.6 | 71.7 | 2.4 | 230.6 | 80.0 |
| Total delivered dose | 1.3 | 125.2 | 43.4 | 1.1 | 106.8 | 37.0 |
| Formulation lost | 0.8 | 81.4 | 28.2 | 1.3 | 123.8 | 43.0 |
| during exhalation | | | | | | |
| Dose Inhalation/ | 1.5 | | | 0.9 | | |
| exhalation ratio | | | | | | |

Replicate measurements (n = 2) performed and average data is presented, as per USP<1601>

### Example 5. Pharmacokinetics and Tissue Distribution of Example Formulations

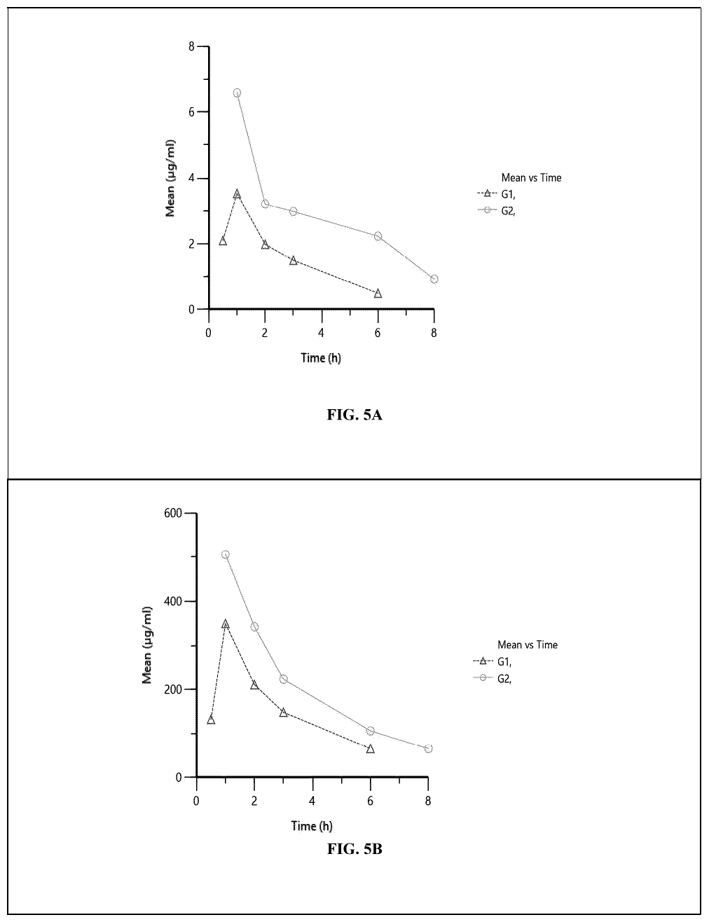

The pharmacokinetics and tissue distribution of metformin was determined in male Wistar rats following a single inhalation exposure for 60 minutes through a nose-only dynamic inhalation chamber. A total of forty male Wistar rats were segregated into two treatment groups of 20 animals each. Groups G1 (5% formulation) and G2 (10% formulation) animals were exposed to the formulations of Example 1 in the form of liquid aerosol through a nose-only dynamic inhalation chamber for a period of 60 minutes. All animals were monitored for survival and clinical signs of toxicity during the exposure period. Body weight was measured prior to treatment. The experimental protocol included collection of samples up to 8 h. At each time, there were three animals for blood and tissue samples. Once the samples were collected plasma and appropriate tissues (viz., liver, kidney, lungs, heart, pancreas, brain and intestine) from each animal were harvested, processed and stored frozen until analyses. A fit-for-purpose LC-MS/MS method was employed for the quantification of metformin levels in plasma or lung homogenized tissue samples. Under the present experimental conditions, the deposited doses following 60-minute inhalation exposure to example 5% formulation (G1) and 10% formulation (G2) were 5.81 and 11.64 mg/kg respectively. Plasma and lung tissue pharmacokinetics for 5% and 10% formulations are shown in FIGS. 5A and 5B, and Table 7 and Table 8.

TABLE 7
Pharmacokinetic parameters for metformin following nose only
inhalation for 60 minutes of 5% and 10% metformin formulations

| | | | Cₘₐₓ | AUC ₀₋ₜ | AUC _{0-inf} | | |
|---|---|---|---|---|---|---|---|
| | | Tₘₐₓ | (μg/ml/ | (μg · h/ml | (ug · h/ml | | T_{1/2} |
| Matrix | Group | (h) | μg/g) | or μg · h/g) | or ug · h/g) | AUC_{% Extra} | (h) |
| Plasma | G1_5% | 1 | 3.52 | 9.37 | 10.76 | 12.87 | 1.96 |
| | G2_10% | 1 | 6.58 | 22.19 | 26.97 | 17.71 | 3.60 |
| Lung | G1_5% | 1 | 349.29 | 931.18 | 1159.03 | 19.66 | 2.41 |
| | G2_10% | 1 | 506.01 | 1622.19 | 1886.87 | 14.03 | 2.82 |

For Lung tissue read as μg/g for Cₘₐₓ and μg · h/g for AUC.

TABLE 8
Tissue to Plasma ratio of 5% and 10% metformin
formulations at various time points

| | | Time Point | | | | |
|---|---|---|---|---|---|---|
| | | 0.5 | 1 | 2 | 3 | 6 |
| G1 | Lung | 131.43 | 349.29 | 210.33 | 147.51 | 65.42 |
| (metformin | Plasma | 2.09 | 3.52 | 1.98 | 1.49 | 0.49 |
| 5%) | L/P ratio | 63 | 99 | 106 | 99 | 134 |
| | | Time Point | | | | |
| | | 1 | 2 | 3 | 6 | 8 |
| G2 | Lung | 506.01 | 341.68 | 222.77 | 105.53 | 65.15 |
| (metformin | Plasma | 6.58 | 3.2 | 2.97 | 2.22 | 0.92 |
| 10%) | L/P ratio | 77 | 107 | 75 | 48 | 71 |

The inhaled metformin formulations of the subject disclosure demonstrate longer retention of metformin in the rat lung tissue. Overall, the pharmacokinetic data indicated that a higher uptake (average lung/plasma ratio of >75-fold) and longer tissue retention (>8 h) of metformin was possible following inhalation dosing. Collectively, the tissue distribution and pharmacokinetic data of metformin is supportive of a profound local delivery of metformin to achieve higher levels for biomarker activation and/or expression changes to allow inhalation administration at desired frequency for the therapy.

### Examples 6. Stability of Example Formulations

The stability of 5% and 10% metformin formulations was evaluated at LTC (25° C./50% RH) and ACC (40° C./75% RH). The formulations were stable for at least for 6 months at accelerated condition (40° C./75% RH). Table 9 and 10 show example data for 10% formulation.

TABLE 9
Stability data for example 10% formulation, LTC

| Batch: 10% metformin | | | |
|---|---|---|---|
| Condition: LTC | Specification | Initial | 18 weeks |
| Appearance | Clear, | Clear, | Clear, |
| | no discoloration | no discoloration | no discoloration |
| Assay of metformin (% LC) | 90-110 | 90.0 | 90.5 |
| Related Substances (% Area) | | | — |
| Impurity A | FIO | 0.01 | 0.02 |
| Impurity D | FIO | n.d. | n.d. |
| Unidentified | FIO | n.d. | n.d. |
| Total impurities | FIO | 0.01 | 0.02 |
| pH | 6.5-8.5 | 6.5 | 7.39 |
| Osmolality (mOsm/kg) | FIO | 1249 | 1251 |

FIO = For information only,
LTC = acc(25° C./50% RH) or at least 6 months at accelerated condition

TABLE 10
Stability data for example 10% formulation, ACC

| Batch: 10% metformin | | | |
|---|---|---|---|
| Condition: ACC | Specification | Initial | 18 weeks |
| Appearance | Clear, | Clear, | Clear, |
| | no discoloration | no discoloration | no discoloration |
| Assay of metformin (% LC) | 90-110 | 90.0 | 90.3 |
| Related Substances (% Area) | | | — |
| Impurity A | FIO | 0.01 | 0.03 |
| Impurity D | FIO | n.d. | 0.01 |
| Unidentified | FIO | n.d. | 0.02 |
| Total impurities | FIO | 0.01 | 0.06 |
| pH | 6.5-8.5 | 6.5 | 8.36 |
| Osmolality (mOsm/kg) | FIO | 1249 | 1258 |

FIO = For information only,
ACC = 40° C./75% RH

### Enumerated Embodiments

### Embodiments (Device)

1) Devices which generate an aerosol for oral inhalation by a subject of a biguanide (e.g., metformin) or a pharmaceutically acceptable salt thereof (e.g., metformin hydrochloride).
2) Devices of embodiment 1 which use a jet of gas as a dispersing force.
3) Devices of embodiment 1 which use an ultrasound wave as a dispersing force.
4) Devices of embodiment 1 which use a vibrating mesh as a dispersing force.
5) Devices of embodiment 1 which rely on propellants to generate the aerosol.
6) Devices of embodiment 1 which rely on impinging jets to generate the aerosol.
7) Devices of embodiment 1 which rely on the Rayleigh instability to generate the aerosol.
8) Devices of embodiment 1 which are breath activated and/or a dry product inhaler.

### Embodiments (Formulations)

A) Formulations for aerosolization of a biguanide (e.g., metformin) or a pharmaceutically acceptable salt thereof (e.g., metformin hydrochloride) using water as vehicle.
B) Formulations for aerosolization of a biguanide (e.g., metformin) or a pharmaceutically acceptable salt thereof (e.g., metformin hydrochloride) containing mixtures of cosolvents and water.
C) Formulations for aerosolization of a biguanide (e.g., metformin) or a pharmaceutically acceptable salt thereof (e.g., metformin hydrochloride) containing hydrofluoroalkanes as solvent and/or propellant.
D) Formulations for aerosolization of a biguanide (e.g., metformin) or a pharmaceutically acceptable salt thereof (e.g., metformin hydrochloride) containing lactose or mannitol as carrier.
E) Formulations of any one of embodiments A-D in which the quality of the ingredients, for which pharmaceutical compendial requirements exist, is met and/or the formulation is suitable for pulmonary administration to a human subject.
F) Formulations of embodiment B wherein the cosolvents include one or more of ethanol, buffers and propylene-glycol.
G) Formulations of any one of embodiments A-D where the metformin is in the form of a metformin salt, where counterion is of known toxicity.
H) Formulations of any one of embodiments A-D in which the salt is hydrochloride.
I) Formulations of any one of embodiments A-D in which the salt is fumarate.
J) Formulations of any one of embodiments A-D in which the salt is succinate.
K) Formulations of any one of embodiment B in which the concentration of the cosolvent is between 1 and 90% v/v.
L) Formulations of any one of embodiments A-K which exhibit an osmolality of not less than 280 mOsm/kg, or from 300 mOsm/kg to 1500 mOsm/kg.
M) Formulations of any one of embodiments A-L which exhibit a pH between 4.0-9.0, for from 7.0-9.0.

### Embodiments (Formulations with Devices)

I. Formulations of any one of embodiments A-M emitted via any one of devices 1 to 8 which generate emitted doses containing between 10-100 mg of metformin per 60 s.
II. Formulations of any one of embodiments A-M emitted via any one of devices 1 to 8 where at least 19% the emitted dose is contained in droplets/particles with aerodynamic sizes<5 microns.
III. Formulations of any one of embodiments A-M emitted via any one of devices 1 to 8 which generate mass median aerodynamic diameters (MMAD) between 1.1 and 7 microns.
IV. Formulations of any one of embodiments A-M emitted via any one of devices 1 to 8 for delivery to lung to treat lung conditions or diseases.
V. Formulations of any one of embodiments A-M emitted via any one of devices 1 to 8 for delivery to lung as an adjuvant in radiation therapy in lung cancers.
VI. Formulations of any one of embodiments A-M emitted via any one of devices 1 to 8 which generate a droplet size distribution obtained by laser diffraction of: d10: 1.5-5.5, d50: 3.5-9.5, d90: 7.0-17.0, span: 1.0-2.0. Span is here defined by (d90-d10)/d50.
VII. Formulations of any one of embodiments A-M emitted via any one of devices 1 to 8 produce 50 to 100 fold differential higher exposure in lungs relative to oral dose, and avoidance or limited exposure of metformin to gastrointestinal tract via drug-device combination.
VIII. Formulations of any one of embodiments A-M emitted via any one of devices 1 to 8 produce long and sustained lung retention to promote biomarker and/or receptor expression changes to aid in therapy.

### Intended Use

a. A method of treating local conditions/diseases in the lung of a subject, such as a sensitizer for standalone radiation therapy or in a concurrent chemotherapy with cytotoxic drugs, or small molecule tyrosine kinase inhibitors or monoclonal antibodies and biological inhibitors in lung cancer including Small Cell Lung Cancer (SCLC) and Non-Small Cell Lung Cancer (NSCLC) patients that includes administering to the subject a formulation of any one of A-M and/or a device of any one of devices 1 to 8.
b. Chemotherapy agents in the embodiment a. include but are not limited to cisplatin, carboplatin and etoposide.
c. Monoclonal antibodies in the embodiment a. include but not are not limited to Cetuximab, Bevacizumab, Nivolumab, Pembrolizumab, Atezolizumab, Ipilimumab.
d. Small molecule tyrosine kinase inhibitors in the embodiment a. include but are not limited to Gefitinib, Erlotinib, Dacomitinib, Osimertinib, Crizotinib, Ceritinib, Lorlatinib.

### REFERENCES

For the sake of brevity, all publications, including patent applications, patents, and other citations mentioned in this document, i.e., both those discussed above and those listed below, are incorporated by reference in their entirety. Citation of any such publication, however, shall not be construed as an admission that it is prior art to the present invention.
Atal, S. and Asokan, P., Recent advances in targeted small-molecule inhibitor therapy for non-small-cell lung cancer—An update J Clin Pharm Ther. 2020; 45:580-584.
Baker S, Dahele M, Lagerwaard F J, Senan S. A critical review of recent developments in radiotherapy for non-small cell lung cancer. Radiat Oncol. 2016 Sep. 6; 11(1):115. doi:10.1186/s13014-016-0693-8.
Brown S L, Kolozsvary A, Isrow D M, Al Feghali K, Lapanowski K. Jenrow K A, Kim J H. A Novel Mechanism of high dose radiation sensitization by metformin. Front Oncol. 2019 Apr. 9; 9:247. doi: 10.3389/fonc.2019.00247.
Choi Y H, Kim S G, Lee M G. Dose-independent pharmacokinetics of metformin in rats: Hepatic and gastrointestinal first-pass effects. J Pharm Sci. 2006 November; 95(11):2543-52. doi: 10.1002/jps.20744.
Dickerson R N, Melnik G. Osmolality of oral drug solutions and suspensions. Am J Hosp Pharm. 1988 April; 45(4):832-4. doi.org/10.1093/ajhp/45.4.832
Duma N, Santana-Davila R, Molina J R. Non-Small Cell Lung Cancer: Epidemiology, Screening, Diagnosis, and Treatment. Mayo Clin Proc. 2019 August; 94(8):1623-1640. doi: 10.1016/j.mayocp.2019.01.013.
Elkins M R, Bye P T. Inhaled hypertonic saline as a therapy for cystic fibrosis. Curr Opin Pulm Med. 2006 November; 12(6):445-52. doi: 10.1097/01.mcp.0000245714.89632.b2.
Fasano M, Della Corte C M, Capuano A, Sasso F C, Papaccio F, Berrino L, Ciardiello F, Morgillo F. A multicenter, open-label phase II study of metformin with erlotinib in second-line therapy of stage IV non-small-cell lung cancer patients: treatment rationale and protocol dynamics of the METAL trial. Clin Lung Cancer. 2015 January; 16(1):57-9. doi: 10.1016/j.cllc.2014.06.010.
Fitzgerald K, Simone C B 2nd. Combining Immunotherapy with Radiation Therapy in Non-Small Cell Lung Cancer. Thorac Surg Clin. 2020 May; 30(2):221-239. doi: 10.1016/j.thorsurg.2020.01.002.
Goldstraw P, Chansky K, Crowley J, Rami-Porta R, Asamura H, Eberhardt W E, Nicholson A G, Groome P, Mitchell A, Bolejack V. The IASLC lung cancer staging project: proposals for revision of the TNM stage groupings in the forthcoming (eighth) edition of the TNM classification for lung cancer. J Thorac Oncol. 2016; 11(1):39-51. doi: 10.1016/j.jtho.2015.09.009.
Graham G G, Punt J, Arora M, Day R O, Doogue M P, Duong J K, Furlong T J, Greenfield J R, Greenup L C, Kirkpatrick C M, Ray J E, Timmins P, Williams K M. Clinical pharmacokinetics of metformin. Clin Pharmacokinet. 2011 February; 50(2):81-98. doi: 10.2165/11534750-000000000-00000.
Hirsch F R, Scagliotti G V, Mulshine J L, Kwon R, Curran W J Jr, Wu Y L, Paz-Ares L. Lung cancer: current therapies and new targeted treatments. Lancet. 2017 Jan. 21; 389(10066):299-311. doi: 10.1016/50140-6736(16)30958-8.
Yousef M, Tsiani E. Metformin in Lung Cancer: Review of in Vitro and in Vivo Animal Studies. Cancers (Basel). 2017 May 6; 9(5):45. doi: 10.3390/cancers9050045.

1.-10. (canceled)

11. A pulmonary delivery system for delivery of a biguanide composition comprising a biguanide or a pharmaceutically acceptable salt thereof in a form suitable to be aerosolized for pulmonary delivery to a human subject and a vibrating mesh nebulizer, wherein at least 19% of the emitted dose of biguanide is contained in droplets or particles having aerodynamic particle sizes of 5 microns or less.

12. The pulmonary delivery system of claim 11, wherein the biguanide is metformin or a pharmaceutically acceptable salt thereof.

13. The pulmonary delivery system of claim 11, wherein the biguanide is metformin hydrochloride.

14. (canceled)

15. (canceled)

16. (canceled)

17. (canceled)

18. (canceled)

19. The pulmonary delivery system of claim 12, wherein the nebulizer includes impinging jets to generate the aerosol.

20. The pulmonary delivery system of claim 12, wherein the nebulizer employs Rayleigh instability to generate the aerosol.

21. The pulmonary delivery system of claim of 11, wherein between about 10 mg to about 100 mg of biguanide is emitted per minute.

22. (canceled)

23. The pulmonary delivery system of claim 11, wherein the emitted dose of biguanide has a mass median aerodynamic diameter (MMAD) of between 1.1 and 7 microns.

24. The pulmonary delivery system of claim 11, wherein the emitted dose of biguanide has a droplet size distribution, as determined by laser diffraction, of:
d10: 1.5-5.5;
d50: 3.5-9.5;
d90: 7.0-17.0.

25. The pulmonary delivery system of claim 24, wherein the span (d90−d10)/d50 is 1.0-2.0.

26. The pulmonary delivery system of claim 11, wherein the administered pulmonary dose provides at least a 50 fold increase in exposure to lung tissue, as compared to the same dose when administered to the same subject orally.

27.-83. (canceled)

84. The pulmonary delivery system of claim 11, wherein the biguanide composition is (a) in the form of a dry powder, (b) in aqueous solution or (c) maintained at an increased pressure.

85. The pulmonary delivery system of claim 84, wherein the biguanide composition is in the form of a dry powder.

86. The pulmonary delivery system of claim 84, wherein the composition is in aqueous solution and further includes one or more cosolvents.

87. The pulmonary delivery system of claim 86, wherein the cosolvents include one or more of ethanol, propylene glycol and a pH buffer.

88. The pulmonary delivery system of claim 84, wherein the composition is in the form of an aqueous solution having an osmolality of at least 280 mOsm/kg.

89. The pulmonary delivery system of claim 84, having a pH between 4.0-9.0.

90. The pulmonary delivery system of claim 84, wherein the composition includes a propellant and is maintained at an increased pressure.

91. The pulmonary delivery system of claim 90, wherein the propellant includes a hydrofluoroalkane.
